(19) United States
(12) Patent Application Publication
(10) Pub. No.: US 2025/0228729 A1
(43) Pub. Date: Jul. 17, 2025

(54) AN ACTUATOR FOR AN EXOSKELETON

(71) Applicants: The Regents of the University of California, Oakland, CA (US); suitX, Inc., Emeryville, CA (US)

(72) Inventors: Wayne TUNG, Emeryville, CA (US); Homayoon KAZEROONI, Oakland, CA (US); Jing-Song HUANG, Emeryville, CA (US); Yi ZENG, Emeryville, CA (US); Zhendong LIU, Emeryville, CA (US)

(73) Assignees: The Regents of the University of California, Oakland, CA (US); suitX, Inc., Emeryville, CA (US)

(21) Appl. No.: 18/854,727

(22) PCT Filed: Apr. 11, 2023
(86) PCT No.: PCT/US2023/018163

§ 371 Date: Oct. 7, 2024

Related U.S. Application Data
(60) Provisional application No. 63/362,778, filed on Apr. 11, 2022.
(60) Provisional application No. 63/362,779, filed on Apr. 11, 2022.

(51) Int. Cl. A61H 3/00; B25J 9/00

(52) U.S. Cl. CPC A61H 3/00; B25J 9/0006; A61H 2201/1621; A61H 2201/5066; A61H 2201/5069

(57) ABSTRACT

An actuator for an exoskeleton can include a motor and a spring. The motor can include a housing and shaft. A first end of the spring can be coupled to one of the shaft or housing. A second end of the spring can be free when the shaft is in a first range of rotation of the shaft relative to the housing. The second end of the spring can be constrained by the other one of the shaft or housing when the shaft is in a second range of rotation of the shaft relative to the housing. When the shaft is in the first range of rotation, the motor can provide a motor resistive torque between the shaft and the housing, and when the shaft is in the second range of rotation, the spring can provide a spring resistive torque between the shaft and the housing.

### CROSS-REFERENCE TO RELATED APPLICATIONS

This application claims the benefit of U.S. Provisional Patent Application No. 63/362,778, filed Apr. 11, 2022, and U.S. Provisional Patent Application No. 63/362,779, filed Apr. 11, 2022. Each of these applications is incorporated herein by reference in its entirety.

International Application No. To Be Assigned (“TRUNK SUPPORT EXOSKELETON WITH ONE POWERED ACTUATOR,” Inventors Wayne TUNG et al., Attorney Docket No. 5085.004PC02), filed on the same day herewith, is incorporated herein in its entirety by reference thereto.

### TECHNICAL FIELD

The present disclosure relates generally to exoskeleton systems and more specifically to powered exoskeleton systems and actuators.

### BACKGROUND

It may be desirable to provide assistive torques to various human joints. Assistive torques can, for example, decrease the likelihood of injuries to joints, which can occur, for example, due to repetitive maneuvers.

### SUMMARY

Some embodiments described herein are directed to an actuator for an exoskeleton. The actuator can include a motor and a spring. The motor can include a motor housing and a motor shaft. A first end of the spring can be coupled to one of the motor shaft or the motor housing. A second end of the spring can be free when the motor shaft is in a first range of rotation of the motor shaft relative to the motor housing. The second end of the spring can be constrained by the other one of the motor shaft or the motor housing when the shaft is in a second range of rotation of the motor shaft relative to the motor housing. In response to a load torque imposed on the motor shaft relative to the motor housing: when the motor shaft is in the first range of rotation, the motor can provide a motor resistive torque between the motor shaft and the motor housing to counteract the load torque, and when the motor shaft is in the second range of rotation, the spring can provide a spring resistive torque between the motor shaft and the motor housing to counteract the load torque.

In some embodiments, in response to a load torque imposed on the motor shaft relative to the motor housing, when the motor shaft is in the first range of rotation, only the motor provides a resistive torque between the motor shaft and the motor housing to counteract the load torque.

In some embodiments, in response to a load torque imposed on the motor shaft relative to the motor housing, when the motor shaft is in the second range of rotation, the motor provides a motor resistive torque between the motor shaft and the motor housing to counteract the load torque.

In some embodiments, in response to a load torque imposed on the motor shaft relative to the motor housing, when the motor shaft is in the second range of rotation, the motor does not provide a motor resistive torque between the motor shaft and the motor housing to counteract the load torque.

In some embodiments, the load torque includes a torque due to the weight of a user's body part.

In some embodiments, the load torque includes a torque due to the weight of a user's trunk.

In some embodiments, the actuator includes a sensor that generates a signal indicating an angle of the motor shaft relative to the motor housing.

In some embodiments, the motor includes an element or combination of elements selected from a group consisting of AC (alternating current) motors, brush-type DC (direct current) motors, brushless DC motors, electronically commutated motors (ECMs), stepper motors, and combinations thereof.

In some embodiments, the spring includes an element or combination of elements selected from a group consisting of coil springs, leaf springs, bungee cords, rotary springs, helical springs, elastomer cords, elastic cords, fabric cords, plastic cords, cord, twine, wire rope elastomers, and string.

In some embodiments, the first end of the spring is coupled to the motor shaft.

In some embodiments, the first end of the spring is coupled to the motor shaft. In some embodiments, the second end of the spring is constrained by a housing constraining element on the motor housing when the motor shaft is in the second range of rotation.

In some embodiments, the location of the housing constraining element is adjustable to alter an angle of the motor shaft relative to the motor housing at which the spring begins providing the spring resistive torque.

In some embodiments, the housing constraining element is configured to be disabled manually.

In some embodiments, the first end of the spring is coupled to the motor housing.

In some embodiments, the first end of the spring is coupled to the motor housing. In some embodiments, the second end of the spring is constrained by a shaft constraining element on the motor shaft when the motor shaft is in the second range of rotation.

In some embodiments, the location of the shaft constraining element is adjustable to alter an angle of the motor shaft relative to the motor housing at which the spring begins providing the spring resistive torque.

In some embodiments, the shaft constraining element is configured to be disabled manually.

Some embodiments described herein are directed to a trunk support exoskeleton actuator configured to be coupled to a trunk support exoskeleton including a supporting trunk frame configured to be coupled to a trunk of a wearer, and a thigh link configured to be coupled one of the wearer's thighs, the thigh link rotatably coupled to the supporting trunk frame such that the thigh link can flex or extend relative to the supporting trunk frame. The actuator can include a motor and a spring. The motor can include a motor housing and a motor shaft. One of the motor shaft or the motor housing can be configured to be coupled to the supporting trunk frame of the trunk support exoskeleton, and the other one of the motor shaft or motor housing can be configured to be coupled to the thigh link of the trunk support exoskeleton. When a wearer is bent forward in a sagittal plane relative to a vertical gravitational line while wearing the trunk support exoskeleton: when the wearer is bent forward in a first bending range, the motor can provide a motor resistive torque between the motor shaft and the motor housing to counteract a torque imposed on the motor shaft relative to the motor housing by a weight of the wearer's trunk, and when the wearer is bent forward in a second bending range, the spring can provide a spring resistive torque between the motor shaft and the motor housing to counteract a torque imposed on the motor shaft relative to the motor housing by a weight of the wearer's trunk.

In some embodiments, the motor shaft is in a first range of rotation of the motor shaft relative to the motor housing when the wearer is bent forward in the first bending range. In some embodiments, the motor shaft is in a second range of rotation of the motor shaft relative to the motor housing when the wearer is bent forward in the second bending range. In some embodiments, a first end of the spring is coupled to one of the motor shaft or the motor housing. In some embodiments, a second end of the spring is free when the motor shaft is in the first range of rotation. In some embodiments, the second end of the spring is constrained by the other one of the motor shaft or the motor housing when the shaft is in the second range of rotation.

In some embodiments, the wearer is bent further forward, relative to the vertical gravitational line, in the second bending range than in the first bending range.

In some embodiments, the motor housing is configured to be coupled to the supporting trunk frame of the trunk support exoskeleton.

In some embodiments, the motor shaft is configured to be coupled to the supporting trunk frame of the trunk support exoskeleton.

In some embodiments, the actuator includes a tilt sensor to generate a tilt signal indicative of an angle of the supporting trunk frame relative to the vertical gravitational line in the sagittal plane.

In some embodiments, the actuator includes a controller to send a signal to the actuator to generate the resistive torque when the tilt signal indicates an angle of the supporting trunk frame relative to the vertical gravitational line that is greater than a predetermined angle.

In some embodiments, the tilt sensor includes an element or combination of elements selected from a group consisting of Inertial Measurement Units (IMU), inclinometers, encoders, and angle sensors.

In some embodiments, the actuator provides a resistive torque that is a function of the tilt signal.

In some embodiments, the actuator includes a controller to send a signal to the actuator to generate the resistive torque when the wearer is bent forward.

In some embodiments, the controller sends a signal to the actuator to generate a substantially small resistive torque between the motor housing and the motor shaft when the wearer is not bent forward.

In some embodiments, the actuator provides a resistive torque that is a function of how much the wearer is bent forward relative to the vertical gravitational line.

In some embodiments, the actuator provides a resistive torque that increases as an angle of the supporting trunk frame relative to the vertical gravitational line increases. In some embodiments, the actuator provides a resistive torque that decreases as an angle of the supporting trunk frame relative to the vertical gravitational line decreases.

In some embodiments, the actuator provides a resistive torque that is a function of an angular velocity of the supporting trunk frame in the sagittal plane.

In some embodiments, the actuator provides a resistive torque that decreases as a forward angular velocity of the supporting trunk frame in the sagittal plane increases.

In some embodiments, the actuator provides a resistive torque that increases as a forward angular velocity of the supporting trunk frame in the sagittal plane decreases.

In some embodiments, the actuator provides a resistive torque that decreases as a backward angular velocity of the supporting trunk frame in the sagittal plane increases.

In some embodiments, the actuator provides a resistive torque that increases as a backward angular velocity of the supporting trunk frame in the sagittal plane decreases.

Some embodiments described herein are directed to a trunk support exoskeleton for reducing muscle forces in a wearer's back during forward lumbar flexion. The trunk support exoskeleton can include a supporting trunk frame, a thigh link, and an actuator. The supporting trunk frame can be configured to be coupled to the wearer's trunk. The thigh link can be configured to be coupled to one of the wearer's thighs and can be rotatably coupled to the supporting trunk frame such that the thigh link can flex or extend relative to the supporting trunk frame. The actuator can include a motor including a motor housing and a motor shaft, and a spring. When the wearer is bent forward in a sagittal plane relative to a vertical gravitational line while wearing the trunk support exoskeleton: when the wearer is bent forward in a first bending range, the motor can provide a resistive torque between the motor shaft and the motor housing, the motor resistive torque causing the supporting trunk frame and thigh link to impose an extension torque between the wearer's trunk and the wearer's thigh, and when the wearer is bent forward in a second bending range, the motor and the spring can provide a resistive torque between the motor shaft and the motor housing, the resistive torque of the motor and the spring causing the supporting trunk frame and thigh link to impose an extension torque between the wearer's trunk and the wearer's thigh.

In some embodiments, a first end of the spring is coupled to one of the shaft or the housing. In some embodiments, a second end of the spring is free when the wearer is bent forward in the first bending range. In some embodiments, the second end of the spring is constrained by the other one of the shaft or the housing when the wearer is bent forward in the second bending range.

### BRIEF DESCRIPTION OF THE DRAWINGS

The accompanying drawings, which are incorporated herein and form a part of the specification, illustrate the present disclosure and, together with the description, further serve to explain the principles thereof and to enable a person skilled in the pertinent art to make and use the same.

FIG. 1 shows a cross-sectional view of an embodiment of an actuator for an exoskeleton, with a second end of a spring of the actuator free.

FIG. 2 shows a perspective view of a motor employed in the actuator of FIG. 1.

FIG. 3 shows a cross-sectional view of the actuator of FIG. 1, with the second end of the spring of the actuator constrained by a housing constraining element.

FIG. 4 shows a cross-sectional view of an embodiment of an actuator for an exoskeleton, where a motor housing of the actuator is employed to rotate a load, and with a second end of a spring of the actuator free.

FIG. 5 shows a cross-sectional view of the actuator of FIG. 4, with the second end of the spring of the actuator constrained by a housing constraining element.

FIG. 6 shows a cross-sectional view of an embodiment of an actuator for an exoskeleton, with a shaft constraining element, and with a second end of a spring of the actuator free.

FIG. 7 shows a cross-sectional view of the actuator of FIG. 6, with the second end of the spring of the actuator constrained by the shaft constraining element.

FIG. 8 shows a plot of the resistive torque the actuator of FIG. 1 generates during use.

FIG. 9 shows a perspective view of a person wearing a trunk support exoskeleton with the actuator of FIG. 1.

FIG. 10 shows a perspective view of the trunk support exoskeleton of FIG. 9.

FIG. 11 shows another perspective view of the trunk support exoskeleton of FIG. 9.

FIG. 12 shows a side view of a wearer wearing the trunk support exoskeleton of FIG. 9, with the wearer bending forward in a sagittal plane.

FIG. 13 shows a side view of a wearer wearing the trunk support exoskeleton of FIG. 9, with the wearer upright in the sagittal plane.

FIG. 14 shows a diagram of forces on a person's back when bending forward in the sagittal plane.

FIG. 15 shows a flow chart of a control algorithm for controlling the trunk support exoskeleton of FIG. 9.

### DETAILED DESCRIPTION

Wearable exoskeletons can be used to reduce forces in various human joints. For example, a trunk support exoskeleton can be used to reduce muscle forces in a wearer's back during forward lumbar flexion. As another example, a knee support exoskeleton can be used to reduce knee joint forces during squatting. As yet another example, a shoulder support exoskeleton can be used to reduce shoulder joint forces.

Some wearable exoskeletons can include a mechanical joint and an actuator that operates the mechanical joint (e.g., by applying a torque to extend or flex the mechanical joint). For example, a trunk support exoskeleton can include a trunk frame, thigh links movably coupled to the trunk frame, and an actuator that applies a torque to extend or flex the thigh links relative to the trunk frame. In use, when a wearer bends forward while wearing the trunk support exoskeleton, the actuator can provide a torque to extend the thigh links relative to the trunk frame and thereby reduce the muscle forces in the wearer's back.

Some embodiments of the present disclosure provide an active actuator (i.e., an actuator that uses an external power source such as a battery) that can be used to operate a mechanical joint of an exoskeleton.

In some embodiments, the actuator includes a motor and a spring that provide a torque (e.g., to a joint of an exoskeleton) either in parallel or alone, depending on a mode of operation. For example, during a first mode of operation, the motor alone can provide a torque. During a second mode of operation, the motor and the spring can both provide a torque.

In some embodiments, the actuator is part of a trunk support exoskeleton. In some such embodiments, the actuator includes a motor and a spring that provide a torque to extend or flex thigh links of the trunk support exoskeleton relative to a trunk frame of the trunk support exoskeleton. In some such embodiments, when a user bends forward while wearing the trunk support exoskeleton, the motor provides a torque to extend the thigh links relative to the trunk frame. In some such embodiments, when a user bends further forward while wearing the trunk support exoskeleton, both the motor and the spring provide a torque to extend the thigh links relative to the trunk frame. In this way, when the user bends further forward, the actuator can provide a greater torque to extend the thigh links relative to the trunk frame than the actuator could provide with a motor torque alone.

These and other embodiments are discussed below in more detail with reference to the figures.

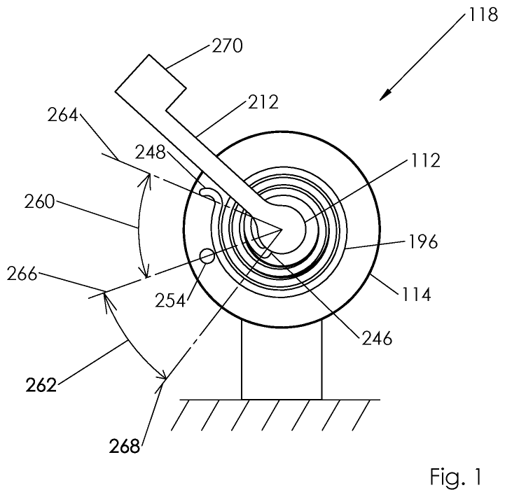

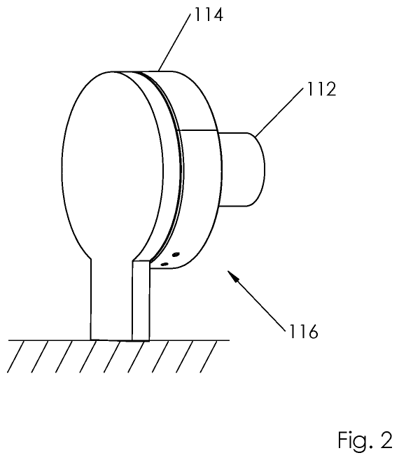

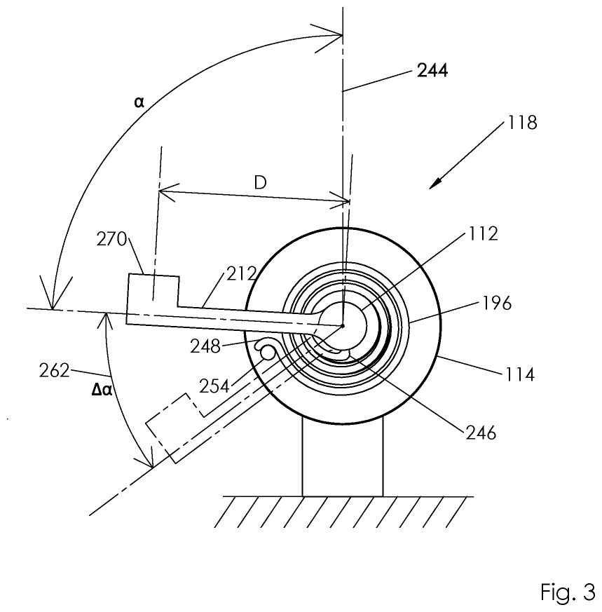

FIGS. 1-7 depict embodiments of an actuator 118 for an exoskeleton. In some embodiments, actuator 118 includes a motor 116 and a spring 196 (shown in FIGS. 1 and 3). Motor 116, as shown in FIG. 2, can include a motor housing 114 and a motor shaft 112. Motor shaft 112 can rotate with respect to motor housing 114. In some embodiments, motor 116 can provide a torque on motor shaft 112 relative to motor housing 114 by use of electric power.

Motor 116 can be or include any device or combination of devices capable of performing the indicated functions. Examples of motor 116 include, electric motors, including, without limitation, AC (alternating current) motors, brush-type DC (direct current) motors, brushless DC motors, electronically commutated motors (ECMs), stepping motors, and combinations thereof. In some embodiments, actuator 118 includes transmission systems such as harmonic drives, planetary gears, ball screw mechanism, lead screw mechanism, worm gear and combinations thereof. In some embodiments, actuator 118 includes hydraulic actuators.

In some embodiments, bar 212 is coupled to motor shaft 112. In turn, bar 212 can be coupled to a load 270 such as, for example, the weight of a portion of an exoskeleton frame and the corresponding portion of the wearer's body such as arms, torso, or legs. It will be described below how actuators 118 shown in FIGS. 1-7 can be used in a trunk support exoskeleton to support the weight of a wearer's trunk during bending or stooping.

As shown in FIGS. 1 and 3, in some embodiments, actuator 118 includes a spring 196. Spring 196 can include a first end 246 and a second end 248. First end 246 of spring 196 can be coupled to motor shaft 112 such that when motor shaft 112 turns relative to motor housing 114 first end 246 of spring 196 turns together with motor shaft 112. In the embodiment shown in FIG. 1, spring 196 is a spiral rotary spring. However, in other embodiments, other types of springs can be employed.

As shown in FIG. 1, a first range of rotation 260 of motor shaft 112 relative to motor housing 114 is defined where second end 248 of spring 196 is not constrained and can rotate with motor shaft 112. As motor shaft 112 rotates (e.g., in a counterclockwise direction), second end 248 of spring 196 also rotates and eventually gets constrained by a housing constraining element 254 at angle 266. Housing constraining element 254 can be coupled to motor housing 114. During this first range of rotation 260 of motor shaft relative to motor housing (when second end 248 of spring 196 is between angles 264 and 266), both spring 196 and motor shaft 112 rotate with the same speed since second end 248 of spring 196 is not constrained.

FIG. 3 shows actuator 118 when second end 248 of spring 196 is constrained by housing constraining element 254. Any rotation of motor shaft 112 with second end 248 of spring 196 constrained occurs in a second range of rotation 262 of motor shaft 112 relative to motor housing 114 (shown in FIG. 1).

FIG. 3 also shows load 270 when it is rotated by Δ∝ after second end 248 of spring 196 is constrained by constraining element 254. When motor shaft 112 is in second range of rotation 262 in which second end 248 of spring 196 is constrained, further rotation of motor shaft 112 relative to motor housing 114 can cause spring 196 to deflect and thereby cause spring 196 to provide a spring resistive torque on motor shaft 112. FIG. 3 shows the second range of rotation 262 of motor shaft 112 which is equivalent to rotational deflection of spring 196. When motor shaft 112 rotates back into first range of rotation 260 (e.g., by rotating in a clockwise direction), second end 248 of spring 196 can lose its contact with housing constraining element 254 (or motor housing 114) and spring 196 stops providing a spring resistive torque to motor shaft 112.

In operation, when motor shaft 112 is in first range of rotation 260, motor 116 can create a motor resistive torque on motor shaft 112 to counteract a load torque on motor shaft 112 relative to motor housing 114. And when motor shaft 112 is in second range of rotation 262, second end 248 of spring 196 can be constrained and therefore spring 196 can provide a spring resistive torque on motor shaft 112 to counteract the load torque. In some embodiments, when motor shaft 112 is in second range of rotation 262, motor 116 can also provide a motor resistive torque on motor shaft 112 thereby increasing the total actuator resistive torque counteracting the load torque. The load torque can include torques due to the gravity and acceleration of load 270. In some embodiments, load torque can include torques due to the external forces and torques on load 270.

In some embodiments, when motor shaft 112 is in first range of rotation 260 only a motor resistive torque counteracts the load torque (i.e., spring 196 does not provide a spring resistive torque to counteract the load torque), but when motor shaft 112 is in second range of rotation 262, spring 196 provides a spring resistive torque and motor 116 provides a motor resistive torque to counteract the load torque. Accordingly, when motor shaft 112 is in the second range, actuator 118 provides more resistive torque than motor 116 alone would provide. Without spring 196, one would need a stronger motor to counteract the same load torque.

In some embodiments, the location of housing constraining element 254 is adjustable. This adjustability allows adjustment of first range of rotation 260 and second range of rotation 262. For example, by changing the location of housing constraining element 254, spring 196 can engage with housing constraining element 254 at a smaller or greater angle of motor shaft 112 relative to motor housing 114, and spring 196 can begin providing a spring resistive torque at a smaller or greater angle of motor shaft 112 relative to motor housing 114.

In some embodiments, housing constraining element 254 can be disabled manually. This can be done, for example, by either pushing or pulling housing constraining element 254 into or out of a cavity manually. When housing constraining element 254 is disabled, the spring 196 does not provide a resistive torque and any actuator resistive torque will solely be provided by motor 116.

In the embodiment shown in FIGS. 1-3, motor shaft 112 rotates counterclockwise relative to motor housing 114 when moving from first range of rotation 260 to second range of rotation 262. However, in other embodiments, motor shaft 112 can rotate clockwise relative to motor housing 114 when moving from first range of rotation 260 to second range of rotation 262.

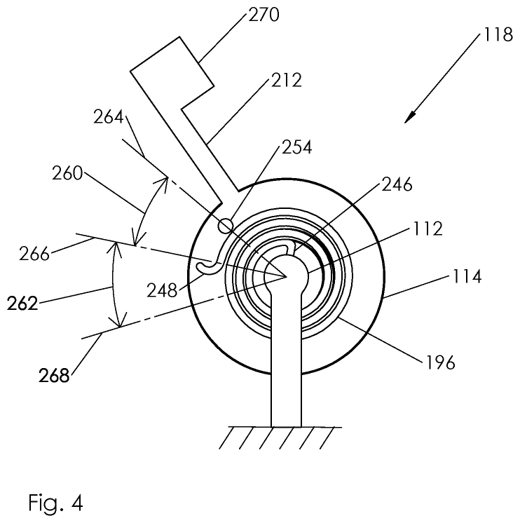

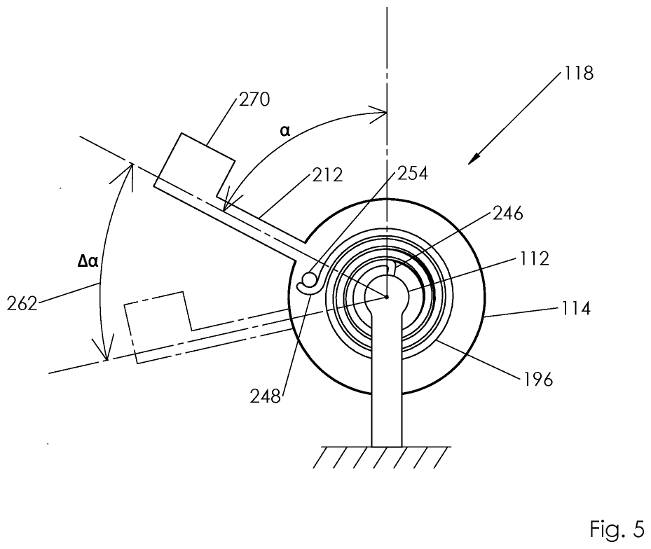

In the embodiment shown in FIGS. 1-3, load 270 is coupled to motor shaft 112. However, in other embodiments, as shown in FIGS. 4 and 5, load 270 can instead be coupled to motor housing 114. As shown in FIG. 4, first range of rotation 260 of motor housing 114 relative to motor shaft 112 is defined where housing constraining element 254 does not contact second end 248 of spring 196. Housing constraining element 254 can be coupled to motor housing 114. When motor housing 114 is in this first range of rotation 260 of motor housing 114 relative to motor shaft 112, where housing constraining element 254 is between angles 264 and 266, motor housing 114 and housing constraining element 254 rotate together toward second end 248 of spring 196.

FIG. 5 shows actuator 118 when housing constraining element 254 has contacted second end 248 of spring 196. Any rotation of motor housing 114 with housing constraining element 254 contacting second end 248 of spring 196 occurs in second range of rotation 262 of motor housing 114 (shown in FIG. 4). FIG. 5 also shows load 270 when it is rotated by Δ∝ after second end 248 of spring 196 is pushed (i.e. constrained) by constraining element 254. When motor housing 114 is in this second range of rotation 262 in which second end 248 of spring 196 is constrained, further rotation of motor housing 114 relative to motor shaft 112 can cause spring 196 to deflect and thereby cause spring 196 to provide a spring resistive torque on motor housing 114. When motor housing 114 rotates back to first range of rotation 260 (e.g., by rotating in a clockwise direction), second end 248 of spring 196 can lose its contact with housing constraining element 254 (or motor housing 114) and spring 196 stops providing a spring resistive torque.

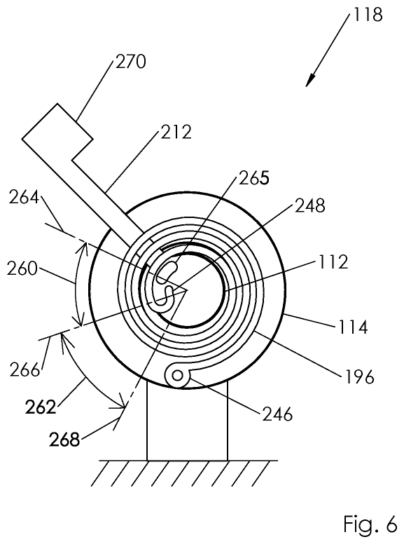

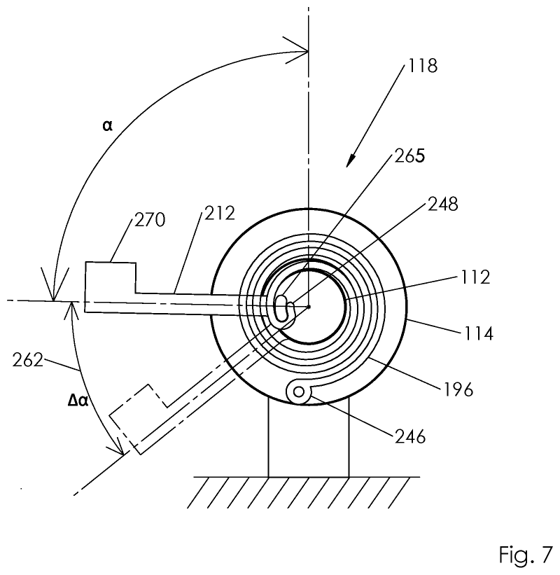

In the embodiment shown in FIGS. 1-5, first end 246 of spring 196 is coupled to motor shaft 112. However, in other embodiments, for example in the embodiment shown in FIGS. 6 and 7, first end 246 end of spring 196 is instead coupled to motor housing 114.

As shown in FIG. 6, first range of rotation 260 of motor shaft 112 relative to motor housing 114 is defined where shaft constraining element 265 is not in contact with second end 248 of spring 196. As motor shaft rotates (e.g., in a counterclockwise direction), shaft constraining element 265 comes in contact with second end 248 of spring 196 at angle 266. During this first range of rotation 260 of motor shaft 112 relative to motor housing 114 (when constraining element 265 is between angles 264 and 266), motor shaft 112 rotates without resistance from spring 196.

FIG. 7 shows actuator 118 when shaft constraining element 265 has contacted second end 248 of spring 196. Any rotation of motor shaft 112 with second end 248 of spring 196 contacting shaft constraining element 265 occurs in second range of rotation 262 of motor shaft 112 relative to motor housing 114. When motor shaft 112 is in this second range of rotation 262 in which second end 248 of spring 196 is pushed by motor shaft 112 (or shaft constraining element 265) further rotation of motor shaft 112 can cause spring 196 to deflect and thereby cause spring 196 to provide a spring resistive torque on motor shaft 112. The spring deflection is shown by A x. When motor shaft 112 rotates back to first range of rotation 260 (e.g., by rotating in a clockwise direction), shaft constraining element 265 can lose its contact with spring 196 and spring 196 stops providing a spring resistive torque to motor shaft 112. Similar to the actuator of FIG. 4, the actuator of FIG. 6 can be used in such a way where motor shaft 112 is stationery and motor housing 114 is employed to rotate load 270.

Based on the embodiments described above it can be observed that, in general, spring 196 includes a first end 246 and a second end 248. First end 246 of spring 196 can be coupled to one of motor shaft 112 or motor housing 114. When motor shaft 112 is in a first range of rotation 260 relative to motor housing 114, second end 248 of spring 196 can be free. Thus, when motor shaft 112 is in first range of rotation 260, spring 196 does not provide a spring resistive torque on motor shaft 112. When motor shaft 112 is in second range of rotation 262 relative to motor housing 114, second end 248 of spring 196 can be constrained by the other one of motor shaft 112 or motor housing 114. In operation, when motor shaft 112 is in first range of rotation 260, motor 116 can provide a motor resistive torque on motor shaft 112 relative to motor housing 114 (e.g. to counteract a load torque imposed on the motor shaft relative to the motor housing). When motor shaft 112 is in second range of rotation 262, spring 196 can provide a spring resistive torque on motor shaft 112 relative to motor housing 114 (e.g., to counteract a load torque imposed on the motor shaft relative to the motor housing). In some embodiments, when motor shaft 112 is in second range of rotation 262, motor 116 can also provide a motor resistive torque on motor shaft 112 relative to motor housing 114 (e.g., to counteract a load torque imposed on the motor shaft relative to the motor housing).

Equation (1) below shows how spring resistive torque and the motor resistive torque T_{M} are provided in parallel with each other and add up to counteract the load torque in quasi static operation.

$${T}_{M} + K Δ ∝ = Mg D Sin ( ∝ ) \qquad ( 1 )$$

Where T_{M} is the motor resistive torque, K is the stiffness of spring 196, Δ∝ is the spring deflection when second end 248 of spring 196 is constrained, Mg is the weight of load 270, K Δ∝ is the spring resistive torque, D is the distance between the center of mass of load 270 and the motor shaft axis of rotation. ∝ is the angle between bar 212 and a vertical gravitational line 244. D and ∝ are shown in FIG. 3. In some embodiments, the spring torque is not a linear function of the spring deflection and in general the spring resistive torque can be expressed as a function of Δ∝ such as ƒ(Δ∝). Equation (1) shows how spring resistive torque and motor resistive torque work in parallel to resist the load torque.

In operation, the motion of motor shaft 112 may not be quasi static (i.e. load 270 may be accelerating or decelerating) and there may be an external torque T_{E} acting on motor shaft 112. Equation (2) represents the behavior of actuator 118 in a more general form.

$$Mg D Sin ( ∝ ) + {T}_{E} - I \ddot{∝} = {T}_{M} + K Δ \ddot{∝} = {T}_{M} + K Δ ∝ \qquad ( 2 )$$

Where \ddot{∝} is the acceleration of motor shaft 112 and/is the moment of inertia of bar 212 and load 270 relative to axis of motor shaft 112. At smaller values of ∝, only motor 116 supports the load torque due to weight, external torque T_{E}, and acceleration (or deceleration) of load 270. At larger values of x, when spring 196 is constrained, the resistive torque of spring 196 will also contribute to counteract load 270 and external torque T_{E}.

When motor shaft 112 is in first range of rotation 260, the motion of motor shaft 112 can be affected by the torque due to the gravity force on load 270 and by the motor resistive torque from motor 116. When motor shaft 112 is in the second range of rotation 262, three torques affect the motion of motor shaft 112: the motor resistive torque from motor 116, the torque due to gravity on load 270, and the spring resistive torque from spring 196.

Actuator 118 of the present disclosure has several advantages:

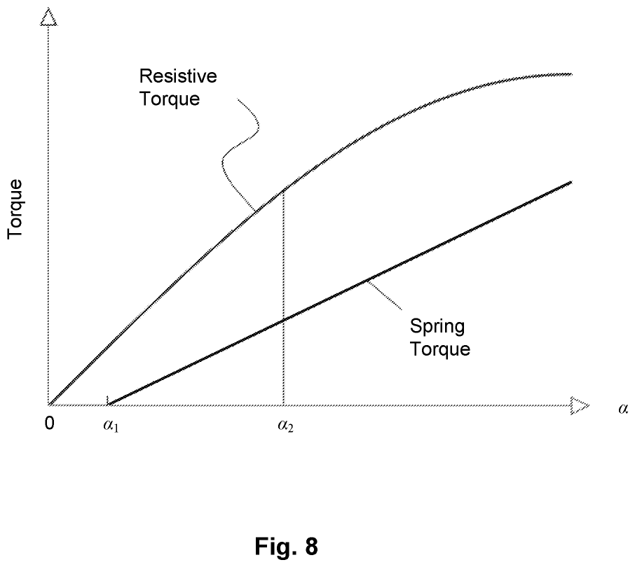

1) As motor shaft 112 rotates and the torque from load 270 increases, spring 196 adds its torque to support the torque of load 270. Without the use of spring 196, one would need a larger motor to provide the required torque to support the weight of load 270. With the actuator described here, which provides a spring torque in parallel with a motor torque, a smaller motor can be utilized. Actuator 118 can be an energy efficient actuator since the size of the motor and batteries can be smaller. FIG. 8 shows an example plot of the total torque actuator 118 can generate in use. As can be seen, at angle ∝₁, spring 196 gets engaged. The difference between these two plots show the torque from motor 116. After ∝₁, (for example at angle ∝₂), the required motor resistive torque Ty is not increased although the total resistive torque is increased.

2) At smaller values of the torque due to gravity on load 270, one can change or adjust the motor resistive torque to provide a desired value of the resistive torque to resist the torque due to gravity on load 270 and external torque T_{E}. This is useful at smaller values of angle ∝. At this region, motor 116 can be programmed to have smooth and seamless transition from a zero value (almost vertical) to a non-zero value. Moreover, the speed of motor shaft 112 can be controlled to utilize various speeds. For example, motor 116 can be controlled to rotate faster along counterclockwise direction than along the clockwise direction. Additionally motor 116 can be configured to provide zero torque (zero impeding torque for the wearer's motion) at smaller angle ∝ where little or no torque is needed. Essentially actuator 118 allows for a more controllable torque when spring 196 is not engaged.

Spring 196 has the characteristic of creating a resisting force or torque in response to deflection passively (i.e. without the use of any power source.) A spring stores energy and subsequently releases it. Examples of actuator spring 196 include, without limitation, coil spring, rotary spring, leaf spring, bungee cord, elastomer cord, elastic cord, elastic fabric cord, plastic cord, elastomer cord, twine, helical spring, tensile spring, wire rope elastomer, string, and combinations thereof.

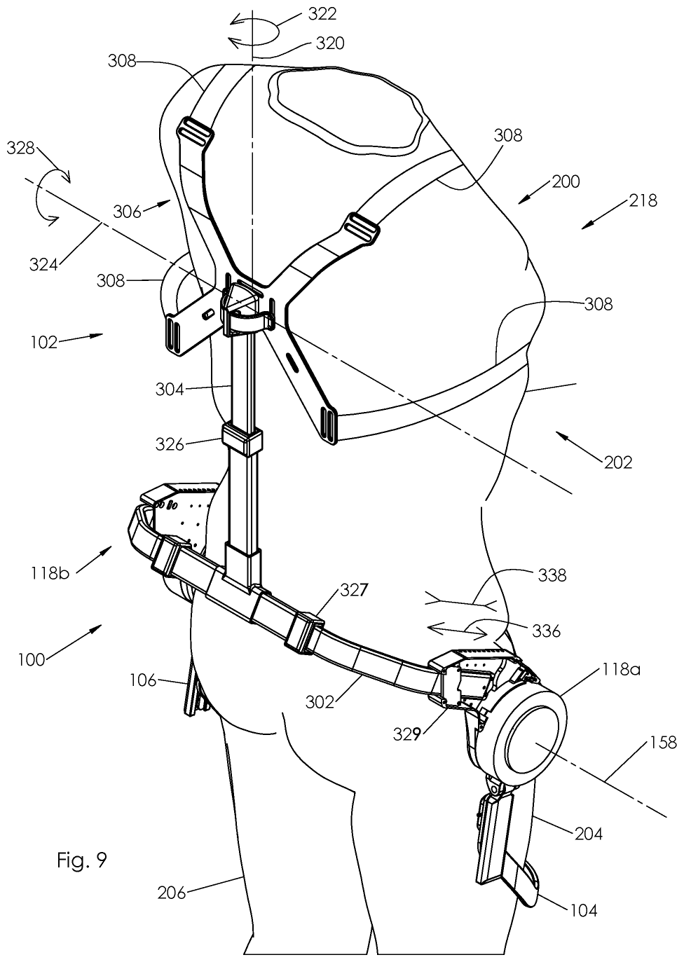

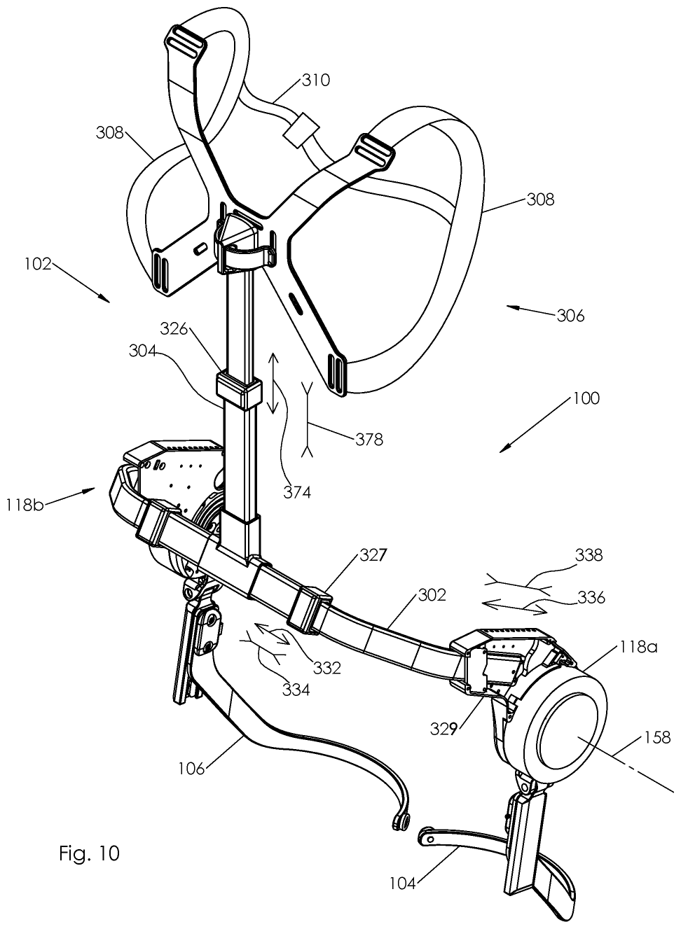

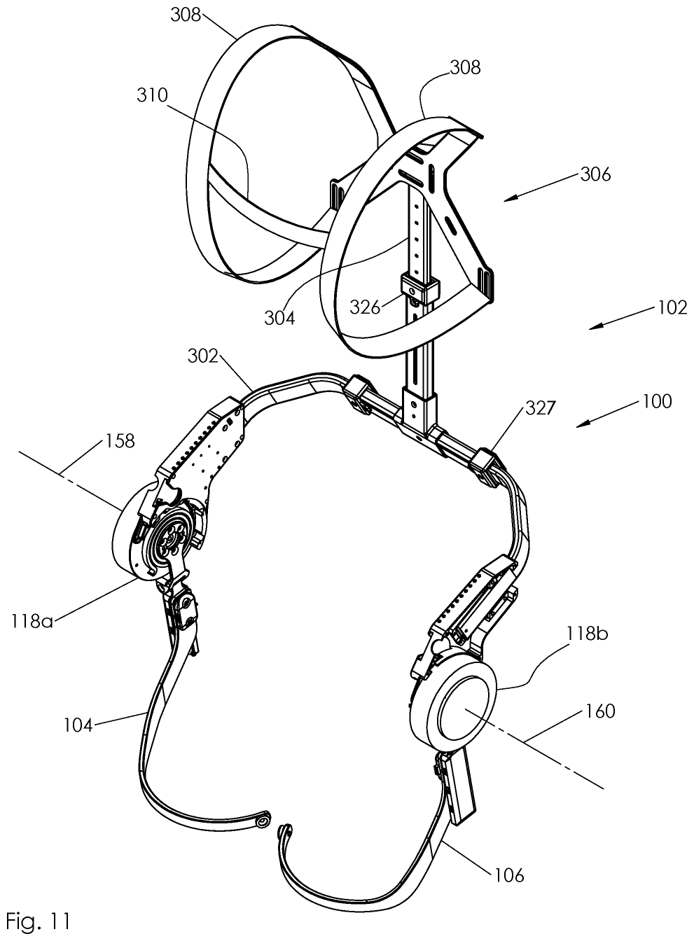

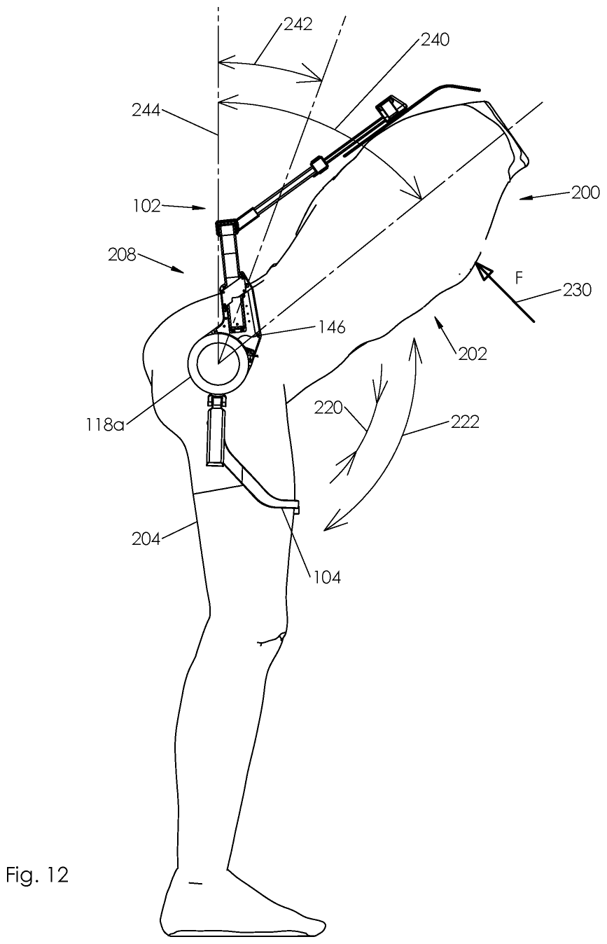

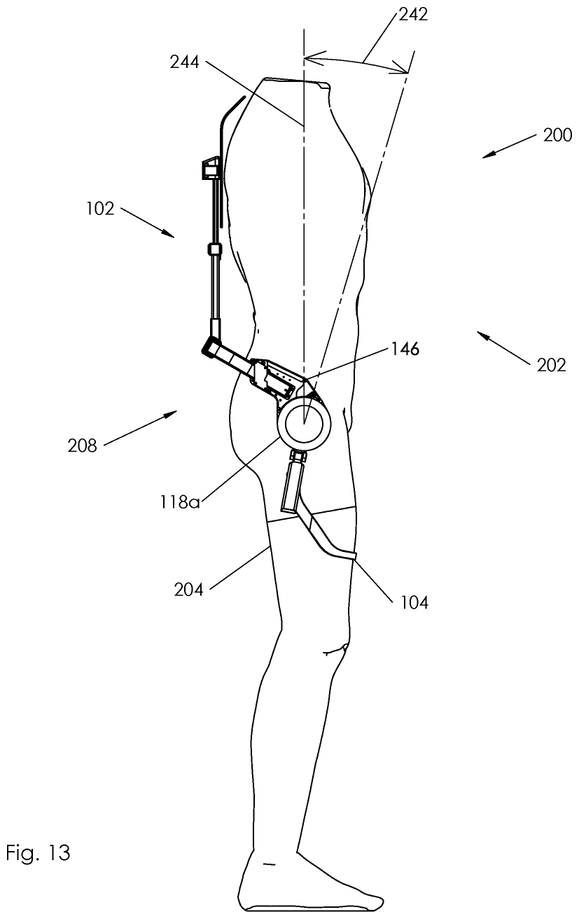

As mentioned, actuator 118 can be used to operate a mechanical joint of an exoskeleton. FIGS. 9-13 show an embodiment of a trunk support exoskeleton 100 including actuator 118. As will be described, trunk support exoskeleton 100 can be worn by a wearer 200 to reduce muscle forces in the wearer's back during forward lumbar flexion which occurs during maneuvers such as stooping and bending. FIG. 9 shows a perspective view of trunk support exoskeleton 100 worn by a wearer 200. FIG. 10 shows a perspective view of trunk support exoskeleton 100 with wearer 200 removed to further illustrate components of trunk support exoskeleton 100. FIG. 11 shows another perspective view of trunk support exoskeleton 100. FIG. 12 shows wearer 200 wearing trunk support exoskeleton 100, bent forward in a sagittal plane. In this position, forward lumbar flexion is taking place. Angle 240 represents how much wearer 200 has bent along the forward direction. FIG. 13 shows wearer 200 wearing trunk support exoskeleton 100 in an upright position in which wearer 200 is not bent forward in the sagittal plane. As shown, for example, in FIG. 9, trunk support exoskeleton 100 can include a supporting trunk frame 102 configured to be coupled to a wearer's trunk 202, a first thigh link 104 configured to be coupled to one thigh 204 of wearer 200, a second thigh link 106 configured to be coupled to another thigh 206 of wearer 200.

As used here and elsewhere in this disclosure, a wearer's trunk 202 can include the wearer's chest, abdomen, pelvis, and back. The wearer's trunk 202 can be, for example, the wearer's body apart from the head and limbs, or the central part of the wearer from which the neck and limbs extend.

In some embodiments, supporting trunk frame 102 includes a lower frame part 302, a spine frame part 304, and an upper frame part 306.

As shown in FIG. 9, in some embodiments, lower frame part 302 is substantially located behind wearer 200 when trunk support exoskeleton 100 is worn. In some embodiments, lower frame part 302 is configured to partially surround wearer's trunk 202 and hips. In some embodiments, lower frame part 302 is coupled to first and second thigh links 104 and 106 from two sides of wearer 200.

Spine frame part 304 can be coupled to (e.g., rotatably coupled to) lower frame part 302. In some embodiments, as shown in FIG. 9, spine frame part 304 is rotatable about axis 320 with respect to lower frame part 302. This can, for example, allow wearer 200 to freely rotate his upper body relative to his lower body. In some embodiments, axis 320 is substantially parallel to the wearer's spine. Arrow 322 shows the direction of rotation of spine frame part 304 relative to lower frame part 302 about axis 320. In some embodiments, supporting spine frame part 304 is located behind wearer 200 when trunk support exoskeleton 100 is worn.

Upper frame part 306, as shown in FIG. 9, can be coupled to (e.g., rotatably coupled to) spine frame part 304. In some embodiments, upper frame part 306 is rotatable about axis 320 with respect to spine frame part 304. This can, for example, allow wearer 200 to freely rotate his upper body relative to his lower body. In some embodiments, axis 320 is substantially parallel the wearer's spine. Arrow 322 shows the direction of rotation of upper frame part 306 relative to spine frame part 304 about axis 320. In some embodiments, as shown in FIG. 9, upper frame part 306 is rotatable about axis 324 relative to spine frame part 304. This can, for example, allow wearer 200 to freely rotate his upper body relative to his lower body. In some embodiments, axis 324 is substantially parallel to one of the wearer's lumbar spine mediolateral flexion and extension axes. Arrow 328 shows the direction of rotation of upper frame part 306 relative to spine frame part 304 about axis 324.

In some embodiments, upper frame part 306 is configured to contact an upper part of wearer's trunk 202 such that upper frame part 306 can impose a force (e.g., supporting trunk force 230 shown in FIG. 12) on a front part of wearer's trunk 202. In some embodiments, upper frame part 306 is configured to contact a chest area of wearer's trunk 202 such that upper frame part 306 can impose a force (e.g., supporting trunk force 230) on a chest area of wearer's trunk 202. In some embodiments, upper frame part 306 is configured to contact a shoulder area 218 of wearer's trunk 202 such that upper frame part 306 can impose a force (e.g., supporting trunk force 230) on a shoulder area 218 of wearer's trunk 202. As shown in FIG. 10, and FIG. 11, in some embodiments, upper frame part 306 includes shoulder straps 308. In some embodiments, as shown in FIG. 10, and FIG. 11, upper frame part 306 includes chest straps 310.

As mentioned, in some embodiments, spine frame part 304 is rotatable with respect to lower frame part 302, and in some embodiments, upper frame part 306 is rotatable with respect to spine frame part 304. In some embodiments, both upper frame part 306 is rotatable with respect to spine frame part 304 and spine frame part 304 is rotatable with respect to lower frame part 302.

In some embodiments, a height of supporting trunk frame 102 is adjustable. In some embodiments, supporting trunk frame 102 includes an adjustment mechanism 326 (shown in FIG. 10) to adjust the height of supporting trunk frame 102. For example, in some embodiments, upper frame part 306 is configured to slide linearly along spine frame part 304 to adjust a height supporting trunk frame 102. In some embodiments, a height of supporting trunk frame 102 can be increased or decreased as shown by arrows 374 and 378 in FIG. 10.

In some embodiments, lower frame part 302 is adjustable in width to fit various people. In some embodiments, supporting trunk frame 102 includes an adjustment mechanism 327 (shown in FIG. 10) to adjust a width of lower frame part 302. For example, in some embodiments, adjustment mechanism 327 can increase or decrease a width of lower frame part 302 as shown by arrows 332 and 334 in FIG. 10.

In some embodiments, lower frame part 302 is adjustable in depth to fit various people. In some embodiments, supporting trunk frame 102 includes an adjustment mechanism 329 (shown in FIG. 9 and FIG. 10) to adjust a width of lower frame part 302. For example, in some embodiments, adjustment mechanism 329 can increase or decrease a depth of lower frame part 302 as shown by arrows 336 and 338 in FIG. 9 and FIG. 10.

As mentioned, and as illustrated in FIG. 9, trunk support exoskeleton 100 can include a first thigh link 104 and a second thigh link 106 which are configured to be coupled to respective thighs 204 and 206 of wearer 200. When first thigh link 104 and second thigh link 106 are coupled to respective thighs 204 and 206, first thigh link 104 and second thigh link 106 can move in unison with wearer's thighs 204 and 206, respectively, in a manner resulting in flexion and extension of respective first and second thigh links 104 and 106 relative to supporting trunk frame 102.

In some embodiments, first and second thigh links 104 and 106 are rotatably coupled to supporting trunk frame 102 such that the first or second thigh links 104 and 106 can flex or extend relative to supporting trunk frame 102. Axes 158 and 160, shown in FIG. 10 and FIG. 11, show the axes of rotation of thigh links 104 and 106 relative to supporting trunk frame 102, respectively. As shown by arrow 220 in FIG. 12, flexion of first thigh link 104 relative to supporting trunk frame 102 occurs when first thigh link 104 and supporting trunk frame 102 rotate towards each other. Similarly, flexion of second thigh link 106 relative to supporting trunk frame 102 occurs when second thigh link 106 and supporting trunk frame 102 rotate towards each other. As shown by arrow 222 in FIG. 12, extension of first thigh link 104 relative to supporting trunk frame 102 occurs when first thigh link 104 and supporting trunk frame 102 rotate away from each other. Similarly, extension of second thigh link 106 relative to supporting trunk frame 102 occurs when second thigh link 106 and supporting trunk frame 102 rotate away from each other.

Trunk support exoskeleton 100 can include a first actuator 118a and a second actuator 118b. First actuator 118a can incorporate some or all of the features discussed above with respect to actuator 118. Second actuator 118b can incorporate some or all of the features discussed above with respect to actuator 118. As shown in FIG. 9 through FIG. 13, in some embodiments, motor housing 114 of each actuator 118a, 118b is coupled to supporting trunk frame 102, and motor shaft 112 of each actuator 118a, 118b is coupled to a respective thigh link 104, 106. In some embodiments, motor shaft 112 of each actuator 118a, 118b is coupled to supporting trunk frame 102, and motor housing 114 of each actuator 118a, 118b is coupled to a respective thigh link 104, 106. First actuator 118a can generate a resistive torque between first thigh link 104 and supporting trunk frame 102. Second actuator 118b can generate a resistive torque between second thigh link 106 and supporting trunk frame 102. In some embodiments, first and second actuators 118a and 118b are located on the right and left halves of wearer 200 substantially close to wearer's hip.

In operation, when wearer 200 is bent forward in a sagittal plane, as shown in FIG. 12, such that a predetermined portion 146 of supporting trunk frame 102 passes beyond a predetermined angle 242 from vertical gravitational line 244, at least one of the first or second actuators 118a and 118b can impose a resisting torque between supporting trunk frame 102 and at least one of the first and second thigh links 104 and 106. This causes supporting trunk frame 102 to impose a supporting trunk force 230 against wearer's trunk 202. In the embodiment of FIG. 12, supporting trunk force 230 is generally imposed on wearer's chest area. However, supporting trunk forces can be imposed on other areas of wearer's trunk 202, as discussed. Supporting trunk force 230 imposed by supporting trunk frame 102 against wearer's trunk 202 helps reduce the muscle forces at the wearer's lower back at the wearer's lower back 208. At the same time, at least one of the first and second thigh links 104 and 106 can impose a force onto wearer's thighs 204 and 206.

In some embodiments, when wearer 200 is bent forward in a first bending range (e.g., bent forward relative to vertical gravitational line 244 between predetermined angle 242 and a toggle angle), motor 116 of at least one of the first or second actuators 118a and 118b can impose a resisting torque between supporting trunk frame 102 and at least one of the first and second thigh links 104 and 106. This causes supporting trunk frame 102 and at least one of the first and second thigh links 104 and 106 to impose an extension torque between the wearer's trunk and the wearer's thigh. For example, motor 116 can provide a motor resistive torque on motor shaft 112 relative to motor housing 114 to counteract a load torque imposed on the motor shaft relative to the motor housing by a weight of the wearer's trunk.

In some embodiments, when wearer 200 is bent forward in a second bending range (e.g., when wearer 200 is bent further forward relative to vertical gravitational line 244, e.g., when wearer 200 is bent forward beyond the toggle angle), spring 196 of at least one of the first or second actuators 118a and 118b can impose a resisting torque between supporting trunk frame 102 and at least one of the first and second thigh links 104 and 106. This causes supporting trunk frame 102 and at least one of the first and second thigh links 104 and 106 to impose an extension torque between the wearer's trunk and the wearer's thigh. For example, spring 196 can provide a spring resistive torque on motor shaft 112 relative to motor housing 114 to counteract a load torque imposed on the motor shaft relative to the motor housing by a weight of the wearer's trunk.

In some embodiments, when wearer 200 is bent forward in the second bending range, motor 116 of at least one of the first or second actuators 118a and 118b can also impose a resisting torque between supporting trunk frame 102 and at least one of the first and second thigh links 104 and 106. For example, motor 116 can also provide a motor resistive torque on motor shaft 112 relative to motor housing 114 to counteract a load torque imposed on the motor shaft relative to the motor housing by a weight of the wearer's trunk.

In some embodiments, when wearer 200 is bent forward in the first bending range, motor shaft 112 is in first range of rotation 260 of the motor shaft 112 relative to motor housing 114 discussed above. In some embodiments, when wearer 200 is bent forward in the second bending range, motor shaft 112 is in second range of rotation 262 of the motor shaft 112 relative to motor housing 114 discussed above.

As shown in FIG. 13, when wearer 200 is not bent forward in the sagittal plane (i.e. when predetermined portion 146 of supporting trunk frame 102 does not pass beyond predetermined angle 242 from vertical gravitational line 244), first and second actuators 118a and 118b, during the entire range of rotation of first and second thigh links 104 and 106, impose no resisting torques between supporting trunk frame 102 and the respective first and second thigh links 104 and 106. This means as long as wearer 200 is not bent forward in the sagittal plane, wearer 200 can walk, ascend and descend stairs and ramps without any force imposed on wearer 200 from supporting trunk frame 102.

However, if wearer 200 bends forward in the sagittal plane (i.e. when a predetermined portion of supporting trunk frame 102 passes beyond predetermined angle 242 from vertical gravitational line 244 as shown in FIG. 12), supporting trunk force 230 from supporting trunk frame 102 will help support wearer's trunk 202.

Examples of predetermined angle 242 can be 5, 10 or 15 degrees. In some embodiments, predetermined angle 242 can be zero.

In some embodiments, when wearer 200 is not bent forward in the sagittal plane, actuators 118a and 118b generate substantially small resistive torques between supporting trunk frame 102 and the respective first and second thigh links 104 and 106. These substantially small resistive torques, generated by actuators 118a and 118b, can cause thigh links 104 and 106 to remain in contact with wearer's thighs during walking. These substantially small resistive torques, generated by actuators 118a and 118b, can be chosen small enough not to resist or impede the wearer during walking, but cause the thigh links to move in unison with the wearer's thighs.

In some embodiments, trunk support exoskeleton 100 includes a controller 170 which sends a signal to first and second actuators 118a and 118b to generate a motor resistive torque (e.g., when wearer 200 is bent forward in the sagittal plane as discussed). Controller 170 can be or include any device or combination of devices capable of performing the indicated functions. Examples of controller 170 include without limitation, analog devices; analog computation modules; digital devices including, without limitation, small-, medium-, and large-scale integrated circuits, application specific integrated circuits, programmable gate arrays, and programmable logic arrays; and digital computation modules including, without limitation, microcomputers, microprocessors, microcontrollers, and programmable logic controllers. In some embodiments controller 170 includes an element or combination of elements selected from a group consisting of electromechanical relays or MOSFET switches.

In some embodiments, trunk support exoskeleton 100 includes a tilt sensor 150 which generates a tilt signal 156. Tilt signal 156 can be indicative of an angle of supporting trunk frame 102 from vertical gravitational line 244 in a sagittal plane. This angle is shown by 240 in FIG. 12.

Tilt sensor 150 can be or include any device or combination of devices capable of performing the indicated functions. Examples of tilt sensor 150 include, without limitation, Inertial Measurement Units (IMU), inclinometers, encoders, and angle sensors.

In operation, controller 170 can send a signal to first and second actuators 118a and 118b to generate a resistive torque when tilt signal 156 indicates that wearer 200 or supporting trunk frame 102 is bent forward in the sagittal plane (i.e., when the tilt signal indicates that an angle of supporting trunk frame 102 from vertical gravitational line 244 in a sagittal plane is greater than predetermined angle 242).

Below an example for calculating the resistive torque of first and second actuators 118a and 118b is described.

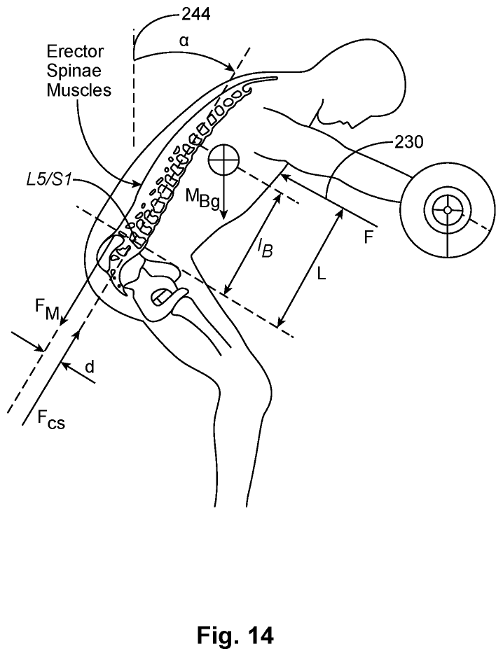

For context, FIG. 14 shows a diagram of forces on a person's back when bending forward in the sagittal plane in the absence of a trunk support exoskeleton. In a static or a quasi-static case and in the absence of any load being lifted by the person, the bending moment (torque) imposed at L5/S1 can be represented by [M_{B} g l_{B} sin(∝)] where M_{B} represents the mass of the person's upper body (including the person's trunk, head and arms), and a part being lifted by person's arms, g represents the gravity acceleration, and l_{B} is the distance between the upper body center of mass and L5/S1 point. ∝ represents the angle of the person's trunk from vertical gravitational line 244. The bending moment increases during load handling and dynamic maneuvers.

As mentioned, when wearer 200 wearing trunk support exoskeleton 100 is bent forward in the sagittal plane, first and second actuators 118a and 118b can create extension torques between supporting trunk frame 102 and first and second thigh links 104 and 106. The extension torques produced by first and second actuators 118a and 118b can produce supporting trunk force 230 onto the wearer opposing the bending moment due to the torso and part weight. This means the bending moment (torque) imposed at L5/S1 can be reduced to a new value: [(M_{B} l_{B}) g sin(∝)−FL] where L is the distance from supporting trunk force 230 to point L5/S1 as shown in FIG. 14. This shows the basic concept of a trunk support exoskeleton, where trunk support exoskeleton decreases the bending moment at L5/S1 and consequently decreases the likelihood of injuries during repetitive maneuvers.

Erector spinae muscle tensile force F_{M} decreases as supporting trunk force 230 increases. In a more general case, erector spinae muscle force F_{M}, with the angular speed and acceleration of \dot{∝} and \ddot{∝}, can be expressed as:

$${F}_{M} = ( {M}_{B} {l}_{B} ) \frac{g}{d} sin ( α ) - \frac{FL}{d} - \ddot{∝} \frac{I}{d} - C \dot{∝} \frac{1}{d} \qquad ( 3 )$$

C is a constant and C \dot{∝} represents the velocity dependent torque. I is the effective moment of inertia of the upper body and \ddot{∝}I represents the acceleration dependent torque.

Spine compression force F_{CS} similarly decreases as supporting trunk force 230 is increased and can be expressed as:

$${F}_{CS} = \frac{g}{d} sin ( ∝ ) + ( {M}_{B} ) g cos ( ∝ ) - ( {M}_{B} {l}_{B} ) {\dot{∝}}^{2} - \ddot{∝} \frac{I}{d} - \dot{∝} \frac{C}{d} - \frac{FL}{d} \qquad ( 4 )$$

This analysis assumes F_{CS} and F_{M} to act perpendicularly to supporting trunk force 230. In theory, if the exoskeleton supporting torque FL is chosen as equation (5), then force F_{M}, reduces to zero (equation 6) and force F_{CS}, reduces substantially (equation 7):

$$F L = ( {M}_{B} {l}_{B} ) g sin ( ∝ ) - I \ddot{∝} - C \dot{∝} \qquad ( 5 )$$

$${F}_{M} = 0 \qquad ( 6 )$$

$${F}_{CS} = ( {M}_{B} ) g cos ( ∝ ) - ( {M}_{B} {l}_{B} ) {\dot{∝}}^{2} \qquad ( 7 )$$

This means, in theory, it is possible to reduce the erector spinae muscle force, F_{M}, to zero and the spine compression force, F_{CS}, to a smaller value by controlling supporting trunk force 230 acting on wearer 200. However, the parameters in equation (5) can be difficult to measure or calculate precisely. For example, M_{B} is not a known quantity, l_{B} is simply estimates, and the measurements of ∝ is not precise. Nevertheless, any attempt to cancel the terms of equation (3) by use of equation (5) will lead to a reduction of the spine compression force, F_{CS} and erector spinae muscle force F_{M}. In most cases, bending acceleration \ddot{∝} is negligible. As these equations demonstrate, when supporting trunk force 230 is increased, both erector spinae muscle force and the spine compression force (shown by F_{CS} and F_{M}) are decreased.

The term F L is referred to as supporting torque because it supports wearer 200 during bending and stooping. As can be seen from FIG. 12, this supporting torque is an extension torque. In some embodiments, exoskeleton supporting torque F L can be chosen as

$$F L = {K}_{1} sin ( ∝ ) - {K}_{2} \dot{∝} \qquad ( 8 )$$

K₁ and, K₂ represent approximate values of parameters of equation (5) if acceleration \ddot{∝} is negligible. As shown by equation (8), the exoskeleton supporting torque FL, in some embodiments, comprises a torque which is a function of angle of ∝. In some embodiments, as shown by equation (8), the exoskeleton supporting torque FL comprises a torque which is a function of the angular speed of the supporting trunk frame \dot{∝}. The acceleration dependent term, I\ddot{∝}, in some applications is small and can be neglected. If can be measured or estimated with little noise, then the inclusion of I\ddot{∝}, in equation (8) can improve the device performance.

The calculated torque for each actuator 118a and 118b, indicated by T_{C}, is represented by equation (9)

$${T}_{C} = \frac{[ F L ]}{2} = [ {K}_{1} sin ( ∝ ) - {K}_{2} \dot{∝} ] / 2 \qquad ( 9 )$$

As long as spring 196 is not constrained (e.g., by housing constraining element 254), then one can use equation (9) to impose a motor resistive torque through first and second actuators 118a and 118b. This means the motor resistive torque for each actuator 118a and 118b (T_{M}) is chosen as

$${T}_{M} = {T}_{C} = [ {K}_{1} sin ( ∝ ) - {K}_{2} \dot{∝} ] / 2 \qquad ( 10 )$$

In some embodiments, when wearer 200 is bent forward in the sagittal plane, actuators 118a and 118b impose a motor resistive torque according to equation (10). As ∝ increases, the relative angle between motor housing 114 and motor shaft 112 of each actuator 118a and 118b changes. Once spring 196 is constrained (e.g., by housing constraining element 254), then the resistive torque comprises the motor resistive torque and the spring resistive torque. This means, if one intends to continue commanding the resistive torque to be as dictated by equation (9), then the resistive torque that needs to be commanded to the motors of first and second actuators 118a and 118b is

$${T}_{M} = {T}_{C} - f ( Δ ∝ ) \qquad ( 11 )$$

Equation (11) indicates that once spring 196 is engaged, the motor resistive torque can be reduced by amount of ƒ(Δ∝) where Δ∝ is the spring deflection and ƒ(Δ∝) is the spring resistive torque due to the spring deflection. Equation (11) implies that although the required resistive torque increases as ∝ increases, one can use a smaller motor torque. This means of a smaller motor can be used for trunk support exoskeletons. In some embodiments, a sensor such as an encoder or a potentiometer in motor 116 can be used to measure the angle between the motor shaft and the motor housing to detect when spring 196 is constrained.

In some embodiments, the calculated resistive torque, as shown by equation (10), is a function of the tilt signal 156. In some embodiments, the calculated resistive torque is a function of how much the wearer is bent forward in the sagittal plane. In some embodiments, the calculated resistive torque increases as the angle of supporting trunk frame 102 from vertical gravitational line 244 increases. In some embodiments, the calculated resistive torque decreases as the angle of supporting trunk frame 102 from vertical gravitational line 244 decreases. In some embodiments, the calculated resistive torque is a function of the angular velocity of supporting trunk frame 102 in the sagittal plane. In some embodiments, the calculated resistive torque decreases as the forward angular velocity of supporting trunk frame 102 in the sagittal plane increases. This can, for example, allow wearer 200 to bend forward in the sagittal plane with little effort to push against supporting trunk frame 102. In some embodiments, the calculated resistive torque increases as the forward angular velocity of supporting trunk frame 102 in the sagittal plane decreases. In some embodiments, the calculated resistive torque decreases as the backward angular velocity of supporting trunk frame 102 in the sagittal plane increases. In some embodiments, the calculated resistive torque increases as the backward angular velocity of supporting trunk frame 102 in the sagittal plane decreases.

In some embodiments, controller 170 stops sending a signal to each actuator 118a and 118b to generate resistive torque according to equation (10) when tilt signal 156 indicates that wearer 200 is no longer bent forward in the sagittal plane.

In some embodiments, controller 170 sends a signal to each actuator 118a and 118b to generate a substantially small resistive torque when tilt signal 156 indicates that wearer 200 is not bent forward in the sagittal plane.

In some embodiments, controller 170 sends a signal to each actuator 118a and 118b to generate a zero torque when tilt signal 156 indicates that wearer 200 is not bent forward in the sagittal plane.

When wearer 200 is not bent forward in the sagittal plane and is walking with long strides, there is a potential that spring 196 can become constrained and provide a spring resistive torque. In some embodiments, first and second actuator 118a and 118b provide an opposing torque (given below) to compensate for the spring resistive torque.

$${T}_{M} = f ( Δ ∝ ) \qquad ( 12 )$$

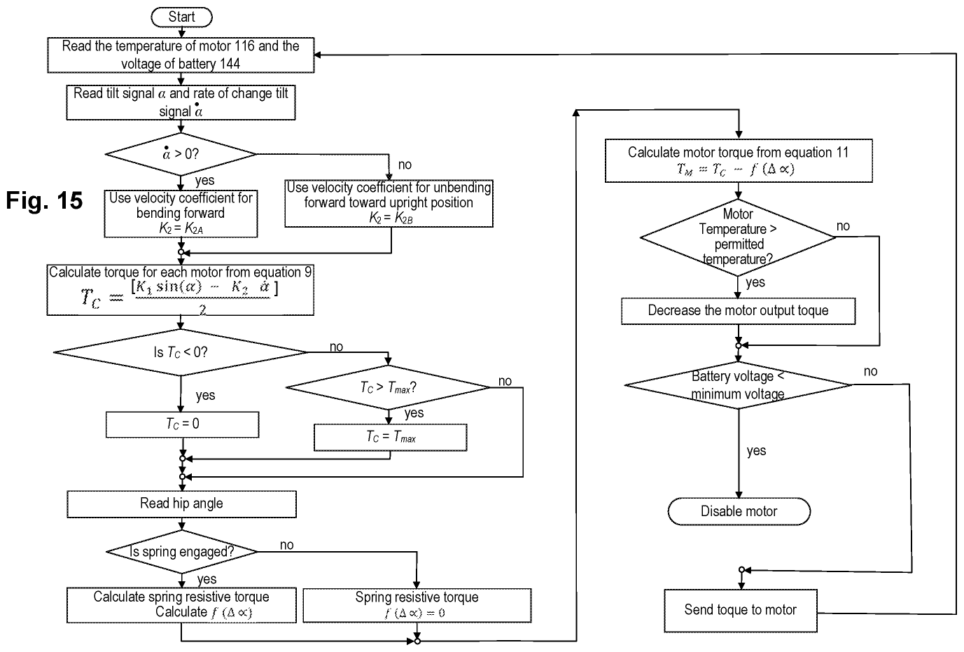

FIG. 15 shows a flowchart of a control algorithm for exoskeleton 100. The control software can start by reading one or more of the voltage of battery 144, the temperature of actuator 118 or a component thereof (e.g., electric motor 116), tilt signal 156, or the rate of change of tilt signal 156.

In embodiments in which the voltage of battery 144 is checked, if the voltage of battery 144 is less than a minimum voltage, motor 116 can be disabled.

In embodiments in which the actuator temperature is checked, if the actuator temperature is larger than a permitted temperature, the motor resistive torque can be decreased.

In embodiments in which tilt signal 156 and/or the rate of change of tilt signal 156 is read, a resistive torque for each actuator can be calculated using equation (9). If \dot{∝} is positive (i.e. wearer 200 is bending forward in the sagittal plane,) coefficient K₂, (used in equation 6) can be K_{2A}. Otherwise, coefficient K₂ can be K_{2B}. In some embodiments, K_{2A} is larger than K_{2B}. This allows the resistive torque to be smaller when bending forward in the sagittal plane. The values of K_{2A} and K_{2B} can be chosen to provide appropriate comfort for wearer 200.

In some embodiments, the calculated resistive torque can be checked to see if it is negative or positive. In some embodiments, if the calculated toque is negative, the calculated resistive torque can be set to zero.

In some embodiments, the calculated resistive torque can be checked to see if it is larger than a maximum toque of electric motor 116. This maximum torque is referred to as Tₘₐₓ. If the calculated value of resisting toque is larger than the maximum torque Tₘₐₓ, then the calculated resistive torque can be chosen as Tₘₐₓ.

In some embodiments, the controller reads the angle between the motor shaft and the motor housing (hip angle) to check if spring 196 is constrained. In some embodiments, the calculated resistive torque will be calculated and adjusted according to equation (11).

1-38. (canceled)

39. An actuator for an exoskeleton, comprising:
a motor comprising a motor housing and a motor shaft; and
a spring, wherein a first end of the spring is coupled to one of the motor shaft or the motor housing, wherein a second end of the spring is free when the motor shaft is in a first range of rotation of the motor shaft relative to the motor housing, and wherein the second end of the spring is constrained by the other one of the motor shaft or the motor housing when the shaft is in a second range of rotation of the motor shaft relative to the motor housing,
wherein, in response to a load torque imposed on the motor shaft relative to the motor housing:
when the motor shaft is in the first range of rotation, the motor provides a motor resistive torque between the motor shaft and the motor housing to counteract the load torque, and
when the motor shaft is in the second range of rotation, the spring provides a spring resistive torque between the motor shaft and the motor housing to counteract the load torque.

40. The actuator of claim 39, wherein in response to a load torque imposed on the motor shaft relative to the motor housing, when the motor shaft is in the first range of rotation, only the motor provides a resistive torque between the motor shaft and the motor housing to counteract the load torque.

41. The actuator of claim 39, wherein in response to a load torque imposed on the motor shaft relative to the motor housing, when the motor shaft is in the second range of rotation, the motor provides a motor resistive torque between the motor shaft and the motor housing to counteract the load torque.

42. The actuator of claim 39, wherein in response to a load torque imposed on the motor shaft relative to the motor housing, when the motor shaft is in the second range of rotation, the motor does not provide a motor resistive torque between the motor shaft and the motor housing to counteract the load torque.

43. The actuator of claim 39, wherein the load torque comprises a torque due to a weight of a user's body part.

44. The actuator of claim 39, further comprising a sensor that generates a signal indicating an angle of the motor shaft relative to the motor housing.

45. The actuator of claim 39, wherein the spring comprises an element or combination of elements selected from a group consisting of coil springs, leaf springs, bungee cords, rotary springs, helical springs, elastomer cords, elastic cords, fabric cords, plastic cords, cord, twine, wire rope elastomers, and string.

46. The actuator of claim 39, wherein the first end of the spring is coupled to the motor shaft.

47. The actuator of claim 39, wherein the first end of the spring is coupled to the motor shaft, and wherein the second end of the spring is constrained by a housing constraining element on the motor housing when the motor shaft is in the second range of rotation.

48. The actuator of claim 47, wherein a location of the housing constraining element is adjustable to alter an angle of the motor shaft relative to the motor housing at which the spring begins providing the spring resistive torque.

49. The actuator of claim 47, wherein the housing constraining element is configured to be disabled manually.

50. The actuator of claim 39, wherein the first end of the spring is coupled to the motor housing.

51. The actuator of claim 39, wherein the first end of the spring is coupled to the motor housing, and wherein the second end of the spring is constrained by a shaft constraining element on the motor shaft when the motor shaft is in the second range of rotation.

52. The actuator of claim 51, wherein a location of the shaft constraining element is adjustable to alter an angle of the motor shaft relative to the motor housing at which the spring begins providing the spring resistive torque.

53. The actuator of claim 51, wherein the shaft constraining element is configured to be disabled manually.

54. An actuator configured to be coupled to a trunk support exoskeleton, the trunk support exoskeleton comprising a supporting trunk frame configured to be coupled to a trunk of a wearer, and a thigh link configured to be coupled one of the wearer's thighs, the thigh link rotatably coupled to the supporting trunk frame such that the thigh link can flex or extend relative to the supporting trunk frame, the actuator comprising:
a motor comprising a motor housing and a motor shaft; and
a spring, wherein one of the motor shaft or the motor housing is configured to be coupled to the supporting trunk frame of the trunk support exoskeleton, and the other one of the motor shaft or motor housing is configured to be coupled to the thigh link of the trunk support exoskeleton,
wherein, when the wearer is bent forward in a sagittal plane relative to a vertical gravitational line while wearing the trunk support exoskeleton:
when the wearer is bent forward in a first bending range, the motor provides a motor resistive torque between the motor shaft and the motor housing to counteract a torque imposed on the motor shaft relative to the motor housing by a weight of the wearer's trunk, and
when the wearer is bent forward in a second bending range, the spring provides a spring resistive torque between the motor shaft and the motor housing to counteract a torque imposed on the motor shaft relative to the motor housing by a weight of the wearer's trunk.

55. The actuator of claim 54, wherein the motor shaft is in a first range of rotation of the motor shaft relative to the motor housing when the wearer is bent forward in the first bending range, wherein the motor shaft is in a second range of rotation of the motor shaft relative to the motor housing when the wearer is bent forward in the second bending range, wherein a first end of the spring is coupled to one of the motor shaft or the motor housing, wherein a second end of the spring is free when the motor shaft is in the first range of rotation, and wherein the second end of the spring is constrained by the other one of the motor shaft or the motor housing when the shaft is in the second range of rotation.

56. The actuator of claim 54, wherein the wearer is bent further forward, relative to the vertical gravitational line, in the second bending range than in the first bending range.

57. The actuator of claim 54, wherein the motor housing is configured to be coupled to the supporting trunk frame of the trunk support exoskeleton.

58. The actuator of claim 54, wherein the motor shaft is configured to be coupled to the supporting trunk frame of the trunk support exoskeleton.
